(19) United States
(12) Patent Application Publication
(10) Pub. No.: US 2008/0183186 A1
(43) Pub. Date: Jul. 31, 2008

(54) METHOD AND APPARATUS FOR DELIVERING A TRANSVASCULAR LEAD

(75) Inventors: Mark J. Bly, Falcon Heights, MN (US); Randy W. Westlund, River Falls, WI (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/669,042

(22) Filed: Jan. 30, 2007

(51) Int. Cl. A61N 1/05

(52) U.S. Cl. 606/129

(57) ABSTRACT

A lead delivery system for delivering a medical electrical lead to an internal jugular vein (IJV) through a subclavian vein. An inner catheter extends from a proximal end to a distal end. The inner catheter includes an inner catheter curve configured to direct the distal end to the IJV when positioned in the subclavian vein. The stiffness of the inner catheter decreases in an inner catheter transition region in a direction from the proximal end to the distal end. An outer catheter extends from a proximal end to a distal end and is sized to slide over the inner catheter. The outer catheter includes an outer catheter curve. The stiffness of the outer catheter decreases in an outer catheter transition region in a direction from the proximal end to the distal end. The system further comprises a guidewire having a distal end and a proximal end. The guidewire is sized to slide through the inner catheter to a desired location in the IJV and the guidewire stiffness decreases in a guidewire transition region in a direction from the guidewire proximal end to the guidewire distal end. A method of delivering a medical electrical lead to a target location within an IJV.

### CROSS-REFERENCE TO RELATED APPLICATIONS

This application is related to the following co-pending and co-owned applications: DUAL SPIRAL LEAD CONFIGURATIONS, filed on the same day and assigned Ser. No. ______; ELECTRODE CONFIGURATIONS FOR TRANSVASCULAR NERVE STIMULATION, filed on the same day and assigned Ser. No. ______; SPIRAL CONFIGURATIONS FOR INTRAVASCULAR LEAD STABILITY, filed on the same day and assigned Ser. No. ______; TRANSVASCULAR LEAD WITH PROXIMAL FORCE RELIEF, filed on the same day and assigned Ser. No. ______; NEUROSTIMULATING LEAD HAVING A STENT-LIKE ANCHOR, filed on the same day and assigned Ser. No. ______; METHOD AND APPARATUS FOR DIRECT DELIVERY OF TRANSVASCULAR LEAD, filed on the same day and assigned Ser. No. ______; and SIDE PORT LEAD DELIVERY SYSTEM, filed on the same day and assigned Ser. No. ______, all herein incorporated by reference in their entirety.

### TECHNICAL FIELD

The present invention relates to delivery systems for medical electrical leads for nerve or muscle stimulation. The present invention more particularly relates to delivery systems and methods of delivering a medical electrical lead into an internal jugular vein and adjacent to a vagus nerve.

### BACKGROUND

A significant amount of research has been directed both to the direct and indirect stimulation of nerves including the left and right vagus nerves, the sympathetic and parasympathetic nerves, the phrenic nerve, the sacral nerve, and the cavernous nerve to treat a wide variety of medical, psychiatric, and neurological disorders or conditions. More recently, stimulation of the vagus nerve has been proposed as a method for treating various heart conditions, including heart failure. Heart failure is a cardiac condition characterized by a deficiency in the ability of the heart to pump blood throughout the body and high filling pressure causing pulmonary fluid to build up in the lungs.

Typically, nerve stimulating electrodes are cuff- or impalement-type electrodes placed in direct contact with the nerve to be stimulated. These electrodes require surgical implantation and can cause irreversible nerve damage due to swelling or direct mechanical damage to the nerve. A less invasive approach is to stimulate the nerve through an adjacent vessel using an intravascular lead. A lead including one or more electrodes is inserted into a patient's vasculature and delivered to a site within a vessel adjacent a nerve to be stimulated.

Standard delivery systems exist for delivering medical electrical leads to regions in or near the heart. Such delivery systems, however, are unsuitable for delivering a medical electrical lead into a patient's internal jugular vein and adjacent to a vagus nerve. Thus, there is a need in the art for a system for delivering a medical electrical lead into the internal jugular vein.

### SUMMARY

In one embodiment, the present invention is a lead delivery system for delivering a medical electrical lead to an internal jugular vein (IJV) through a subclavian vein. The system comprises an inner catheter extending from a proximal end to a distal end. The inner catheter includes an inner catheter curve configured to direct the distal end to the IJV when positioned in the subclavian vein. The stiffness of the inner catheter decreases in an inner catheter transition region in a direction from the proximal end to the distal end. An outer catheter extends from a proximal end to a distal end and is sized to slide over the inner catheter. The outer catheter includes an outer catheter curve. The stiffness of the outer catheter decreases in an outer catheter transition region in a direction from the proximal end to the distal end. The system further comprises a guidewire having a distal end and a proximal end. The guidewire is sized to slide through the inner catheter to a desired location in the IJV and the guidewire stiffness decreases in a guidewire transition region in a direction from the guidewire proximal end to the guidewire distal end.

In another embodiment, the present invention is a lead delivery system for delivering a medical electrical lead to an internal jugular vein (IJV) through a subclavian vein. The system comprises an inner catheter extending from a proximal end to a distal end. The inner catheter includes a curve configured to direct the distal end to the IJV when positioned in the subclavian vein. An outer catheter extends from a proximal end to a distal end and is sized to slide over the inner catheter. A guidewire has a distal end and a proximal end. The guidewire is sized to slide through the inner catheter to a desired location in the IJV.

In another embodiment, the present invention is a method of delivering a medical electrical lead to a target location in an internal jugular vein (IJV) through a subclavian vein. The method comprises inserting an inner catheter through a portion of the subclavian vein and into the IJV. The inner catheter extends from a proximal end to a distal end and includes a curve. A guidewire is inserted through the inner catheter to a desired location in the IJV. The inner catheter is advanced over the guidewire. An outer catheter is advanced over the inner catheter to a desired location in the IJV. The inner catheter is removed. A medical electrical lead is advanced through the outer catheter to a target location in the IJV.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic view of a patient's upper torso.

FIG. 2 shows a side view of an outer catheter for use in a delivery system according to one embodiment of the present invention.

FIGS. 3A-3B show side views of inner catheters for use in a delivery system according to various embodiments of the present invention.

FIG. 4 shows a side view of a guidewire for use in a delivery system according to one embodiment of the present invention.

FIG. 5 shows a schematic view of an inner catheter with its tip located in the right brachiocephalic vein according to one embodiment of the present invention.

FIG. 6 shows a schematic view of a guidewire inserted into an inner catheter according to one embodiment of the present invention.

FIG. 7 shows a schematic view of an inner catheter and guidewire advanced into the internal jugular vein according to one embodiment of the present invention.

FIG. 8 shows a schematic view of an outer catheter, a guidewire, and an inner catheter according to one embodiment of the present invention.

FIG. 9 is a flowchart illustrating an exemplary method of implanting a medical electrical lead into an internal jugular vein according to one embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

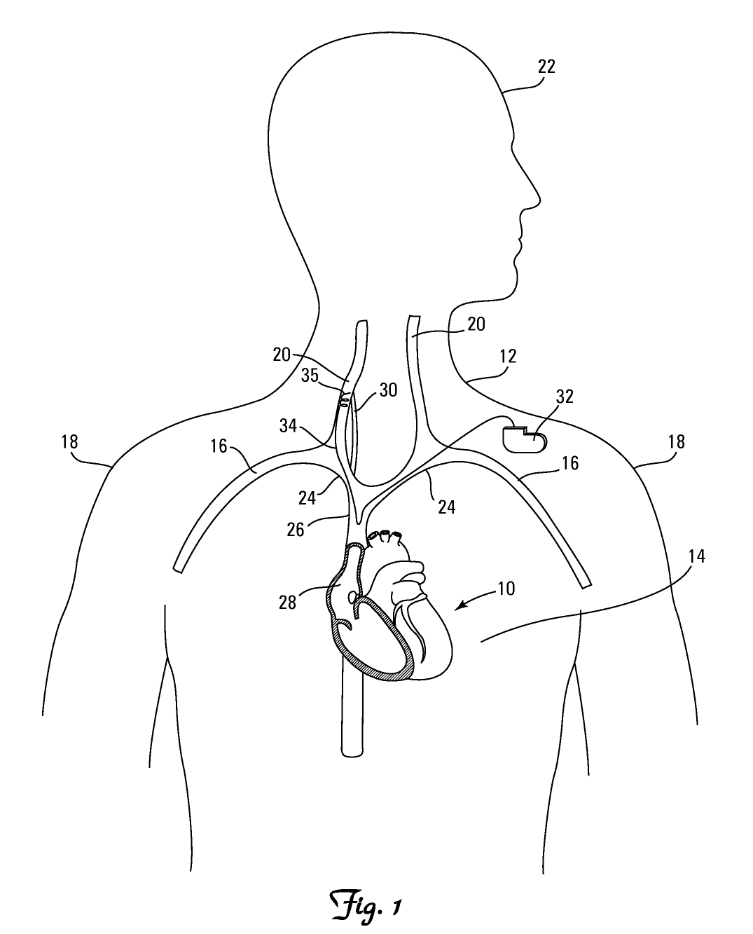

FIG. 1 shows a schematic view of a patient's upper torso, including a heart 10 and the veins of the neck 12 and thorax 14. The subclavian veins 16 drain blood from the arms 18. The internal jugular veins 20 drain blood from the head 22 and join the subclavian veins 16 to form the brachiocephalic or innominate veins 24. The union of the brachiocephalic veins 24 forms the superior vena cava 26, which returns blood from the head 22, neck 12, arms 18, and thorax 14 to the right atrium 28. A vagus nerve 30 is adjacent to the right internal jugular vein 20. Another vagus nerve (not shown) is adjacent to the left internal jugular vein 20. A stimulating device 32 is located in a subcutaneous pocket near the patient's subclavian vein. The stimulating device 32 is connected to a medical electrical lead 34 extending through the patient's subclavian, brachiocephalic, and internal jugular veins. In the illustrated embodiment, the lead 34 includes a retaining structure 35 positioned in the internal jugular vein 20. In one embodiment, the stimulating device 32 provides electrical stimulation to a nerve. In another embodiment, the stimulating device 32 provides electrical stimulation to a vagus nerve 30.

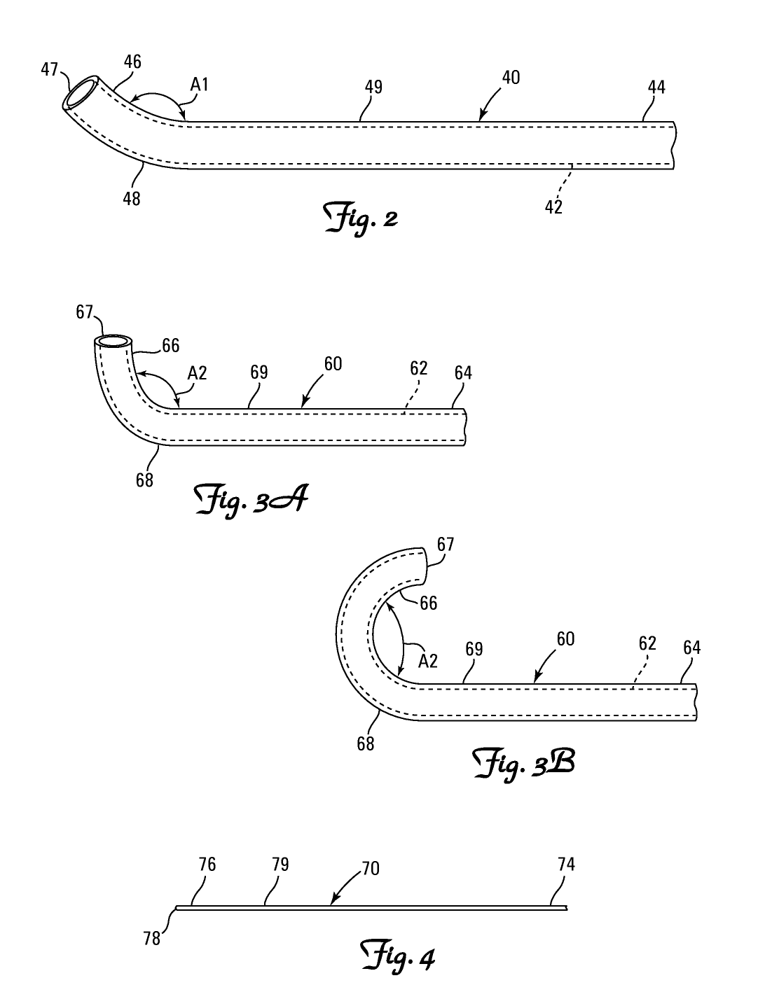

FIG. 2 illustrates an outer catheter 40 according to one embodiment of the present invention. The outer catheter 40 has a lumen 42 extending from a proximal end 44 to a distal end 46. A distal tip 47 is located at the distal end 46. In the illustrated embodiment, the outer catheter 40 includes a curve or bend 48 near the distal end 46. The curve 48 has an angle A1. In one embodiment, the angle A1 is between approximately 0 and approximately 90 degrees. In one embodiment, the outer catheter 40 has an outer diameter of between approximately 6 and approximately 14 French, and an inner diameter slightly less than the outer diameter. In one embodiment, the outer catheter 40 has a length of between approximately 20 and approximately 60 centimeters. In another embodiment, the outer catheter 40 has a length of between approximately 25 and approximately 35 centimeters. In yet another embodiment, the outer catheter 40 has a length of between approximately 30 and approximately 40 centimeters.

FIG. 3A illustrates an inner catheter 60 according to one embodiment of the present invention. The inner catheter 60 includes a lumen 62, which extends from a proximal end 64 to a distal end 66. In the illustrated embodiment, the inner catheter 60 has an “L” shape. In the embodiment illustrated in FIG. 3B, the inner catheter 60 has a “J” shape. In the embodiments shown in FIGS. 3A and 3B, the catheter 60 includes a curve 68 having an angle A2. In one embodiment, the angle A2 is between approximately 40 and approximately 120 degrees. In one embodiment, the inner catheter 60 has an outer diameter of between approximately 4 and approximately 12 French, and an inner diameter slightly less than the outer diameter. In one embodiment, the length of the inner catheter 60 is between approximately 30 and approximately 80 centimeters, and the distance between the curve 68 and the distal tip 67 is between approximately 1 and approximately 2 centimeters. In one embodiment, the curve 68 is configured to direct the distal end 66 to the internal jugular vein 20 when the inner catheter 60 is positioned in the subclavian vein 16. In another embodiment, the curve 68 is configured to select the brachiocephalic vein 24 and to direct the distal end 66 to the internal jugular vein 20 when positioned in the subclavian vein 16.

The inner and outer catheters 40, 60 can be comprised of a polytetrafluoroethylene (PTFE) or fluoronated ethylene propylene (FEP) inner lining, a 304 V stainless steel braiding, and an outer jacket of Pebax and/or Nylon. Tungsten wire can optionally be added to the stainless steel braiding to improve radiopacity of the braiding. In other embodiments, the inner and outer catheters 60, 40 are comprised of any other material known in the art. In one embodiment, the inner braiding does not extend to the distal tips 47, 67, but instead terminates between approximately 4 and 5 millimeters from the distal tips 47, 67, resulting in atraumatic tips 47, 67.

In one embodiment, both of the outer and inner catheters 40, 60 have a constant stiffness from the proximal ends 44, 64 to the distal ends 46, 66. In other embodiments, either the inner catheter 40, the outer catheter 60, or both catheters 40, 60, include a transition region 49, 69 where the stiffness decreases from the proximal ends 44, 64 to the distal ends 46, 66. In one embodiment, the catheters 40, 60 have a Pebax outer jacket (not shown) where the durometer of the outer jacket at the proximal ends 44, 64 is approximately 75D and the durometer of the distal ends 46, 66 is approximately 35D.

In one embodiment, the transition regions 49, 69 include multiple discrete segments having different stiffnesses. In another embodiment, the decrease in stiffness occurs continuously along the transition regions 49, 69. In one embodiment, the transition regions 49, 69 have lengths of between approximately 5 and approximately 20 centimeters. In another embodiment, the total length of the transition regions 49, 69 is between approximately 5 and approximately 15 centimeters. In one embodiment, the transition regions 49, 69 begin at the distal tips 47, 67 and extend proximally approximately 20 centimeters from the distal tips 47, 67. In another embodiment, the transition regions 49, 69 begin at the distal tips 47, 67, and extend proximally between approximately 7 and approximately 10 centimeters from the distal tips 47, 67. In one embodiment, the transition regions 49, 69 include between 3 and 6 segments of decreasing durometers. In another embodiment, the transition regions 49, 69 include segments of decreasing durometers having lengths of between approximately 2 and approximately 7.5 centimeters.

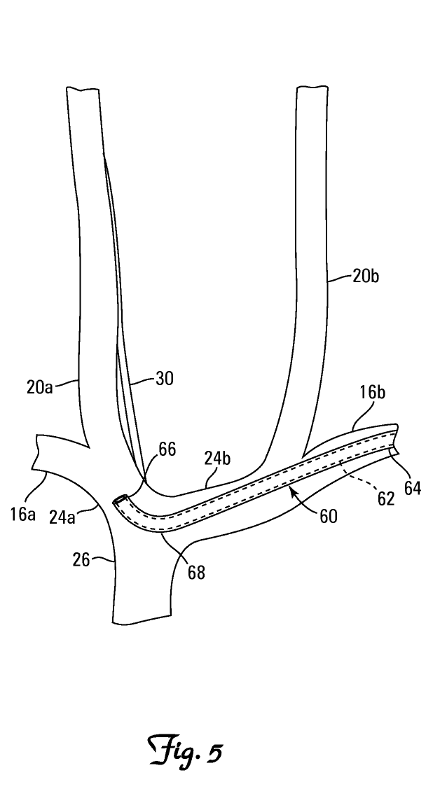

FIG. 4 depicts a guidewire 70 according to one embodiment of the present invention. In the illustrated embodiment, the guidewire 70 includes a proximal end 74, a distal end 76, and a distal tip 78. The guidewire 70 allows a clinician to introduce and position a catheter or a medical electrical lead 34 in a patient. In one embodiment, the guidewire 70 has a core (not shown) and includes a coating, for example, a hydrophilic coating. In one embodiment, the proximal end 74 has a diameter of between approximately 0.012 and approximately 0.040 inch. In one embodiment, the guidewire 70 has a diameter of approximately 0.014 inch. In another embodiment, the guidewire 70 has a diameter of approximately 0.035 inch. In one embodiment, the guidewire 70 includes a grind profile. In one embodiment, the grind profile is parabolic. Although a guidewire 70 is shown in FIG. 4, in other embodiments, a stylet could be used in conjunction with the catheters 40, 60. In another embodiment, a guidewire 70 is used to insert the catheters 40, 60, and a stylet is used to implant the medical electrical lead 34. Although a substantially straight guidewire 70 is depicted in FIG. 4, in other embodiments the guidewire 70 has a J shape.

In one embodiment, the guidewire 70 includes a transition region 79 where the stiffness decreases in a direction from the proximal end 74 to the distal tip 79. In one embodiment, the transition region 79 includes multiple discrete segments having different stiffnesses. In another embodiment, the decrease in stiffness occurs continuously along the transition region 79. In one embodiment, the guidewire stiffness transition is accomplished by providing tapered core segments having different diameters and degrees of taper. In yet another embodiment, the guidewire stiffness transition is accomplished using contiguous tapered core sections as described in U.S. Pat. No. 6,390,993, herein incorporated by reference in its entirety. In another embodiment, the guidewire stiffness transition is accomplished as described in U.S. Pat. No. 6,669,652, herein incorporated by reference in its entirety, by using an elongated core member having a proximal core section, a distal core section and a coil. In this embodiment, the coil has a tapered distal portion with a tapered distal end, is disposed about the distal core section of the core member, and is secured at the distal end to the distal core section. A polymer coating covers only the tapered distal portion.

In one embodiment, the guidewire 70 has a length of between approximately 100 and approximately 250 centimeters. In another embodiment, the transition region 79 has a length of between approximately 10 and approximately 40 centimeters. In one embodiment, the transition region 79 includes between 3 and 6 segments of decreasing stiffness, where each segment is between approximately 1 and approximately 10 centimeters in length. In one embodiment, the transition region 79 extends proximally from the distal tip 78 approximately 20 centimeters.

FIG. 5 is a schematic view showing advancement of the inner catheter 60 through the left subclavian and brachiocephalic veins 16b, 24b and into the right brachiocephalic vein 24a. Although the method of implantation is described as an “opposite side method” from the left subclavian vein 16b into the right internal jugular vein 20a, in alternative embodiments, the method of implantation can comprise implantation from the right subclavian vein 16a into the left internal jugular vein 20b. In other embodiments, the method of implantation is a “same side” implantation from the right subclavian vein 16a into the right internal jugular vein 20a, or the left subclavian vein 16b into the left internal jugular vein 20b. In one embodiment, the inner catheter 60 is inserted into the left subclavian vein 16b using a percutaneous venipuncture. In an alternative embodiment, the inner catheter 60 could be inserted using a surgical cut-down to a subclavian vein 16 from a subcutaneous pocket (not shown) created for the stimulating device 32, or in any other manner known in the art.

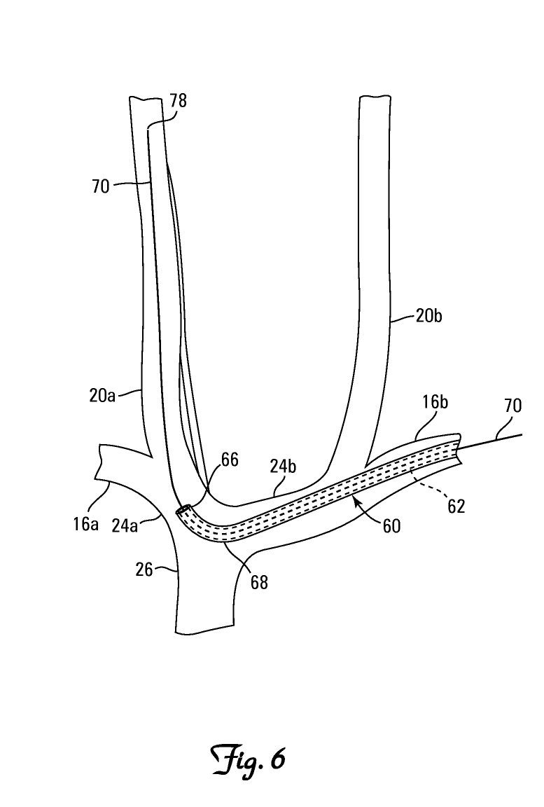

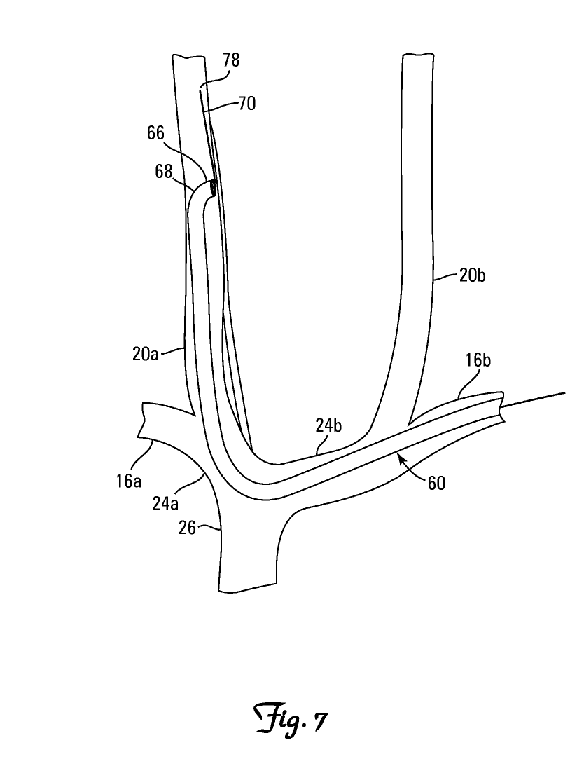

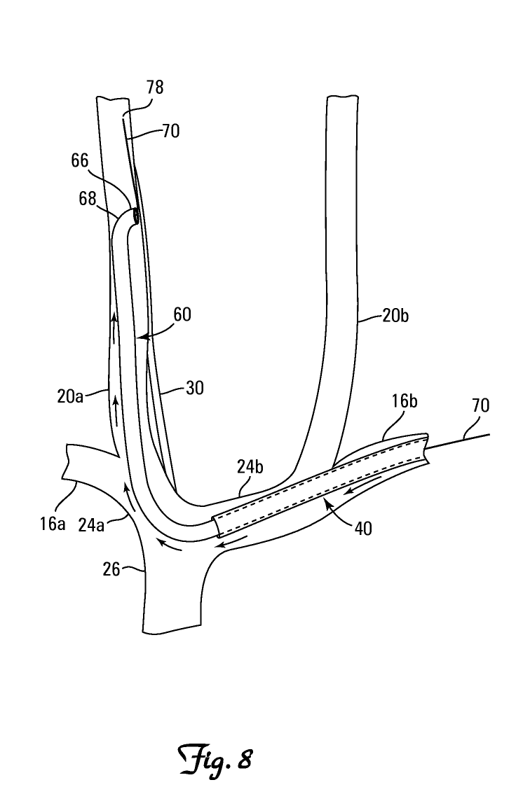

FIG. 6 is a schematic view showing the guidewire 70 after insertion through the lumen 62 of the inner catheter 60. As can be seen in FIG. 6, the inner catheter curve 68 facilitates the advancement of the guidewire distal tip 78 into the right internal jugular vein 20a. FIG. 7 illustrates the inner catheter 60 after it has been advanced over the guidewire 70 to a desired location in the internal jugular vein 20a. FIG. 8 illustrates the advancement of the outer catheter 40 over the inner catheter 70 into the internal jugular vein 20a in the direction shown by the arrows.

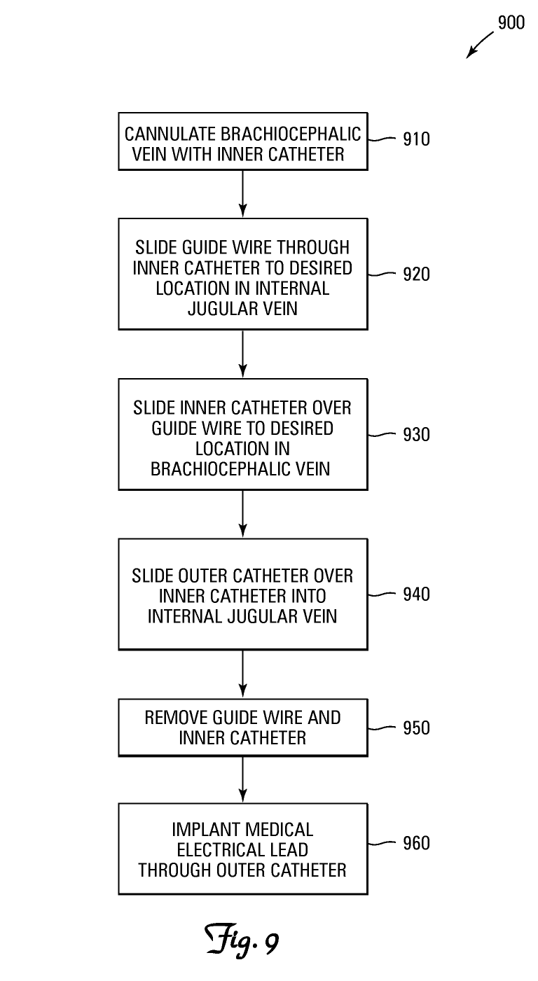

FIG. 9 is a flowchart illustrating an exemplary method 900 of implanting a medical electrical lead 34 in an internal jugular vein 20 from a brachiocephalic vein 22. The inner catheter 60 is used to cannulate the brachiocephalic vein (block 910). In one embodiment, the inner catheter 60 is inserted into the subclavian vein 16 using a percutaneous venipuncture and advanced to the brachiocephalic vein 20. The guidewire 70 is advanced through the lumen 62 of the inner catheter 60 to a desired location in the internal jugular vein 20 (block 920). The inner catheter 60 is advanced over and supported by the guidewire 70 into the internal jugular vein 20 (block 930). The outer catheter 40 is advanced over and supported by the inner catheter 60 to a desired location in the internal jugular vein 20 (block 940). The guidewire 70 and inner catheter 60 are removed (block 950). In one embodiment, the inner catheter 60 is removed by sliding it out of the veins. In another embodiment, the inner catheter 60 comprises a splittable or peelable catheter and is divided into two segments, thereby facilitating removal. A medical electrical lead 34 is advanced through the outer catheter 40 to a target location in the internal jugular vein 20 (block 960). In one embodiment, the target location is adjacent to a vagus nerve 30. In another embodiment, the guidewire 70 is not removed prior to implanting the medical electrical lead 34, and the medical electrical lead 34 is advanced over the guidewire 70 to the target location using an over-the-wire technique. In yet another embodiment, venograms are taken through either the inner or the outer catheters 60, 40 during implantation. In another embodiment, the method is a “same side” method and the inner catheter 60 is inserted directly into the internal jugular vein 20 from the subclavian vein 16.

The medical electrical lead 34 includes an electrode (not shown). In one embodiment, the electrode is located on the retaining structure 35. In one embodiment, the electrode has the form disclosed in U.S. patent application Ser. No. ______, filed ______, 2007, entitled ELECTRODE CONFIGURATIONS FOR TRANSVASCULAR NERVE STIMULATION, above-incorporated by reference in its entirety. In one embodiment, the medical electrical lead 34 and retaining structure 35 have the form disclosed in U.S. patent application Ser. No. ______, filed ______, 2007, entitled SPIRAL CONFIGURATIONS FOR INTRAVASCULAR LEAD STABILITY, above-incorporated by reference in its entirety. In an alternative embodiment, the medical electrical lead 34 and retaining structure 35 have the form of a dual spiral as disclosed in U.S. patent application Ser. No. ______, filed ______, 2007, entitled DUAL SPIRAL LEAD CONFIGURATIONS, above-incorporated by reference in its entirety. In another embodiment, the medical electrical lead 34 and retaining structure 35 have the form disclosed in U.S. patent application Ser. No. ______, filed ______, 2007, entitled NEUROSTIMULATING LEAD HAVING A STENT-LIKE ANCHOR, above-incorporated by reference in its entirety.

The transition regions 69, 79 facilitate advancement of the inner catheter 60 and guidewire 70 around vein junctions, such as the junction between the brachiocephalic vein 24 and the superior vena cava 26 or the subclavian and internal jugular veins 16, 20. The transition region 49 allows the outer catheter 40 to follow the inner catheter 60 and guidewire 70 through the corners and junctions of the veins of the neck 12 and thorax 14. The transition region 49 also reduces kinking of the outer catheter 40 and facilitates delivery of the medical electrical lead 34. In one embodiment, when the guidewire 70 is inserted into the lead 34, the guidewire 70 reduces the force exerted by the retaining structure 35 on a surface external to the retaining structure, for example, the outer catheter 40 or the internal jugular vein 20, thereby facilitating advancement and orientation of the lead 34. In another embodiment, when a portion of the retaining structure remains in the outer catheter 60, the outer catheter 60 is used to rotate the lead 34 and position the electrode proximal to a vagus nerve 30.

Although the outer catheter 40, inner catheter 60, and guidewire 70 have been described are described with respect to the subclavian, brachiocephalic, and internal jugular veins 16, 24, 20, in other embodiments, the delivery system can be used to access other bodily vessels. For example, the delivery system can used to position a medical electrical lead 34 in the subclavian vein, superior vena cava, or azygous vein. The delivery system can be used to position a lead in any vein, artery, lymphatic duct, bile duct, or any other bodily vessel.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A lead delivery system for delivering a medical electrical lead to an internal jugular vein (IJV) through a subclavian vein, the system comprising:
an inner catheter extending from a proximal end to a distal end, the inner catheter including an inner catheter curve configured to direct the distal end to the IJV when positioned in the subclavian vein, wherein the stiffness of the inner catheter decreases in an inner catheter transition region in a direction from the proximal end to the distal end;
an outer catheter extending from a proximal end to a distal end and sized to slide over the inner catheter and including an outer catheter curve, wherein the stiffness of the outer catheter decreases in an outer catheter transition region in a direction from the proximal end to the distal end; and
a guidewire having a distal end and a proximal end;
wherein the guidewire is sized to slide through the inner catheter to a desired location in the IJV and the guidewire stiffness decreases in a guidewire transition region in a direction from the guidewire proximal end to the guidewire distal end.

2. The delivery system of claim 1 wherein the inner catheter curve is further configured to select the brachiocephalic vein when positioned in the subclavian vein.

3. The delivery system of claim 1 wherein the guidewire has a diameter of between approximately 0.012 and 0.040 inch.

4. The delivery system of claim 1 wherein the guidewire has a length of between approximately 100 and approximately 250 centimeters.

5. The delivery system of claim 1 wherein the guidewire transition region has a length of between approximately 10 and approximately 40 centimeters.

6. The delivery system of claim 1 wherein the guidewire transition region extends proximally from a guidewire distal tip approximately 20 centimeters and includes between 3 and 6 segments of decreasing stiffness, each segment having a length of between approximately 1 and approximately 10 centimeters.

7. A lead delivery system for delivering a medical electrical lead to an internal jugular vein (IJV) through a subclavian vein, the system comprising:
an inner catheter extending from a proximal end to a distal end, the inner catheter including an inner catheter curve configured to direct the distal end to IJV when positioned in the subclavian vein;
an outer catheter extending from a proximal end to a distal end and sized to slide over the inner catheter; and
a guidewire having a distal end and a proximal end;
wherein the guidewire is sized to slide through the inner catheter to a desired location in the IJV.

8. The delivery system of claim 7 wherein the inner catheter curve is further configured to select the brachiocephalic vein when positioned in the subclavian vein.

9. The delivery system of claim 7 wherein the inner catheter curve has an angle of between approximately 40 and approximately 120 degrees and the inner catheter curve is located between approximately 1 and 2 centimeters from a distal tip of the inner catheter.

10. The delivery system of claim 7 wherein the inner catheter has an outer diameter of between approximately 4 and approximately 12 French.

11. The delivery system of claim 7 wherein the outer catheter includes a outer catheter curve having an angle of between approximately 0 and approximately 90 degrees.

12. The delivery system of claim 7 wherein the outer catheter has an outer diameter of between approximately 6 and approximately 14 French.

13. The delivery system of claim 7 wherein the stiffness of at least one of the inner catheter and outer catheter decreases in a catheter transition region in a direction from the proximal end to the distal end.

14. The delivery system of claim 13 wherein the transition region has a length of between approximately 5 and approximately 20 centimeters.

15. The delivery system of claim 13 wherein the proximal end has a durometer of approximately 75D and the distal end has a durometer of approximately 35D.

16. The delivery system of claim 13 wherein the stiffness decreases continuously along the catheter transition region.

17. The delivery system of claim 13 wherein the catheter transition region includes between 3 and 6 segments of decreasing stiffness, each segment having a length of between approximately 2 and 7.5 centimeters.

18. A method of delivering a medical electrical lead to a target location in an internal jugular vein (IJV) through a subclavian vein, the method comprising:
inserting an inner catheter through a portion of the subclavian vein and into the IJV, the inner catheter extending from a proximal end to a distal end and including a curve;
inserting a guidewire through the inner catheter to a desired location in the IJV;
advancing the inner catheter over the guidewire;
advancing an outer catheter over the inner catheter to a desired location in the IJV;
removing the inner catheter; and
advancing a medical electrical lead through the outer catheter to a target location in the IJV.

19. The method of claim 18 wherein the method further comprises inserting the inner catheter directly into the IJV from the subclavian vein.

20. The method of claim 18 wherein the method further comprises inserting the inner catheter through the brachiocephalic vein and into the IJV.

21. The method of claim 18 wherein the lead includes an electrode and a retaining structure, and the method further comprises rotating the outer catheter when a portion of the retaining structure remains in the outer catheter to position the electrode proximal to a vagus nerve.

22. The method of claim 18 wherein the method further comprises removing the guidewire before advancing the medical electrical lead.

23. The method of claim 18 wherein advancing the medical electrical lead comprises advancing the medical electrical lead over the guidewire.

24. The method of claim 23 wherein the lead includes a retaining structure and the guidewire reduces a force exerted by the retaining structure on a surface external to the retaining structure.
